(19) United States
(12) Patent Application Publication
(10) Pub. No.: US 2007/0161694 A1
(43) Pub. Date: Jul. 12, 2007

(54) Use of indirubin as a dietary supplement

(76) Inventor: Eric Marchewitz, Howell, MI (US)

Correspondence Address: Miller, Canfield, Paddock and Stone P.L.C.;c/o Robert Kelley Roth, Suite 2500, 150 West Jefferson Ave., Detroit, MI, 48226, US

(21) Appl. No.: 11/652,409

(22) Filed: Jan. 10, 2007

Related U.S. Application Data
(60) Provisional application No. 60/757,778, filed on Jan. 10, 2006.

(51) Int. Cl. A61K 31/404

(52) U.S. Cl. 514/414

(57) ABSTRACT

A method is disclosed for administering naturally occurring chemical Indirubin or its derivatives such as, for example, Indirubin-3′-monoxime, as a method of increasing skeletal muscle mass, decreasing skeletal muscle breakdown and/or decreasing fat mass.

### RELATED APPLICATION

This application claims priority benefit of U.S. provisional patent application No. 60/757,778 filed on Jan. 10, 2006.

### FIELD OF INVENTION

The present invention provides a method of using naturally occurring chemical Indirubin or its derivatives such as, for example, indirubin-3′-monoxime for increasing skeletal muscle mass, decreasing skeletal muscle breakdown and/or decreasing fat mass.

### BACKGROUND OF THE INVENTION

Skeletal muscle mass is an important determinant of health and well-being. Increases in skeletal muscle are associated with improved insulin response, reduced blood glucose and triglycerides and reduced adiposity Improvements in skeletal muscle mass are also associated with increased self-esteem.

There currently are several strategies for increasing muscle mass through the use of pharmaceuticals and dietary supplements. Androgens, in particular, are one of the primary pharmaceutical and supplemental methods for inducing skeletal muscle growth. The use of androgens, while shown to be effective, are associated with increased risk of side effects including gynecomastia, prostate enlargement, atherosclerosis, increased blood pressure and baldness.

The present invention relates to the method of administering the naturally occurring chemical Indirubin or its derivatives, such as, for example, Indirubin-3′-monoxime, for increasing skeletal muscle mass, decreasing skeletal muscle breakdown and/or decreasing fat mass. Indirubin and its derivatives have been shown to be effective inhibitors of the enzyme, glycogen synthase kinase 3-beta (GSK-3B). GSK-3B plays and important role in skeletal muscle metabolism and inhibition of this enzyme has been shown to result in increased skeletal muscle protein synthesis, reduced skeletal muscle catabolism and increased skeletal muscle anabolism. Administering Indirubin or its derivatives is a significant improvement over the use of androgens for increasing skeletal muscle mass, decreasing skeletal muscle breakdown and decreasing fat mass while reducing side effects.

It would be desirable to provide new compounds which can be used to help increase skeletal muscle mass, decreasing skeletal muscle breakdown and/or decreasing fat mass.

### SUMMARY OF THE INVENTION

This invention relates to the use of the naturally occurring chemical indirubin or its derivatives such as, for example, Indirubin-3′-monoxime, as a method of increasing skeletal muscle mass, decreasing skeletal muscle breakdown and/or decreasing fat mass. This invention relates to the use of indirubin, its derivatives and any salts, esters and ethers thereof in animals or humans.

### DETAILED DESCRIPTION OF CERTAIN PREFERRED EMBODIMENTS

It will be apparent to those skilled in the art, that is, to those who have knowledge or experience in this area of technology that many variations are possible for the method of administering, Indirubin or its derivatives is to be administered to an individual in order to increase skeletal muscle mass, decrease skeletal muscle breakdown and/or decrease fat mass. The compound might be administered orally and a typical variety of oral dosage forms might be used. For example, tablets might be prepared by combining the components of the present invention with conventionally used binders and excipients. In one variation the administration amount to an average 70 kilo individual might be about 50-1000 mg per day. A preferable dose might be about 100-250 mg. Unit dosage administration forms might contain about 50-200 mg. A preferable dose might be 100 mg of the compound. Daily oral dosage forms might be administered in smaller divided doses to maintain constant blood levels.

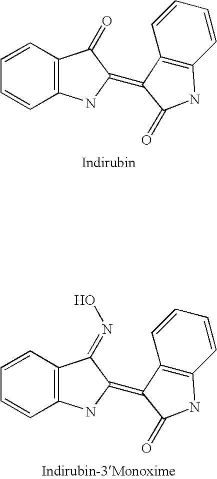

In addition to oral administration, Indirubin or its derivatives might be delivered through sublingual, transdermal, transrectal (by suppository) or intranasal administration. It might also be delivered by intravenous, subcutaneous, or intramuscular injection.

### EXAMPLE 1

Capsules. 25 kg of the Isatis Indigotica leaf, a source of Indirubin was mixed with 25 kg of I-leucine, and placed into 100,000 hard-gelatin capsules each with a content of 250 mg of Isatis and 250 mg of I-leucine.

### EXAMPLE 2

Capsules. 25 kg lsatis Indigotica leaf, a source of Indirubin, was mixed with 25 kg of creatine ethyl ester, and placed into 100,000 hard-gelatin capsules each with a content of 250 mg of Isatis and 250 mg of creatine ethyl ester.

While the fundamental features of the novel nature of the invention have been disclosed herein it should be understood that various aspects of the invention may be made by those skilled in the art without departing from the spirit and scope of the invention. Accordingly, all such modifications or variations are included in the scope of the invention as defined by the claims.

What is claimed is:

1. A method for increasing skeletal muscle mass, decreasing skeletal muscle breakdown and/or decreasing fat mass by the administration of indirubin.

2. The method of claim 1 wherein the compound administered comprises an Indirubin derivative.

3. The method of claim 1 wherein the compound administered comprises the Indirubin derivative Indirubin-3′-monoxime.

4. The method of claim 1 wherein the quantity administered comprises a daily dosage between 50-1000 mg per day.

5. The method of claim 1 wherein the quantity administered is in unit doses of 50 to 250 mg per day.

6. The method of claim 1 wherein the quantity administered is in unit doses of 100 to 200 mg per day.

7. The method of claim 1 wherein the quantity administered is in unit doses of 100 mg per day.

8. The method of claim 7 wherein the quantity administered is in unit doses of 50 mg twice a day.

9. The method of claim 7 wherein the quantity administered is in unit doses of 25 mg four times a day.

10. The method of claim 1 wherein the quantity is administered orally.

11. The method of claim 1 wherein the quantity is administered in the form of a gel capsule.

12. The method of claim 1 wherein the quantity is administered in the form of a liquid suspension.

13. The method of claim 1 wherein the quantity is administered by sublingual delivery.

14. The method of claim 1 wherein the quantity is administered by transdermal delivery

15. The method of claim 1 wherein the quantity is administered by transrectal (by suppository) delivery.

16. The method of claim 1 wherein the quantity is administered by intranasal delivery.

17. The method of claim 1 wherein the quantity is administered by injection.
